(19) United States
(12) Patent Application Publication
(10) Pub. No.: US 2014/0081415 A1
(43) Pub. Date: Mar. 20, 2014

(54) MICROVASCULAR ANASTOMOTIC COUPLER AND METHODS OF USING SAME

(71) Applicants: Jeffrey W. Ruberti, Lexington, MA (US); David Schecter, Boston, MA (US); Nicholas Cote, Derry, NH (US); Ryan Myers, Spencerport, NY (US); Jessica Patel, Tolland, CT (US); Matthew Ouellette, Salem, NH (US); Lifei Guo, Brookline, MA (US)

(72) Inventors: Jeffrey W. Ruberti, Lexington, MA (US); David Schecter, Boston, MA (US); Nicholas Cote, Derry, NH (US); Ryan Myers, Spencerport, NY (US); Jessica Patel, Tolland, CT (US); Matthew Ouellette, Salem, NH (US); Lifei Guo, Brookline, MA (US)

(73) Assignee: The Brigham and Women's Hospital, Inc., Boston, MA (US)

(21) Appl. No.: 13/991,522

(22) PCT Filed: Dec. 7, 2011
(86) PCT No.: PCT/US2011/063759

§ 371 Date: Dec. 4, 2013

Related U.S. Application Data
(60) Provisional application No. 61/420,442, filed on Dec. 7, 2010.

(51) Int. Cl. A61B 17/11; B29C 70/68

(52) U.S. Cl. CPC A61B 17/11; B29C 70/688
USPC 623/23.7; 264/271.1

(57) ABSTRACT

Disclosed herein are devices and methods for fast and simple generation of an anastomosis. In certain embodiments, the devices and methods involve the deployment of self-expanding stents without the use of a catheter. The devices and methods disclosed herein further utilize a sheath that allows an operator to deploy the stent without the use of an inter-lumen device.

This application claims the benefit of priority of U.S. Provisional Application No. 61/420,442, filed Dec. 7, 2010, the entire disclosure of which is incorporated herein by reference.

### FIELD OF THE INVENTION

The invention is generally directed to methods and devices in the field of medicine. More specifically, the invention is related to devices and methods for performing microvascular surgery.

### BACKGROUND OF THE INVENTION

A single anastomosis can take 60-120 minutes, increasing the total surgery time by hours. This lengthy and difficult operation consumes hospital time and resources, in addition to fatiguing surgeons. In military settings, patients requiring microvascular reconstruction often end up with limb amputations due to lack of time or specialized surgeons in the area.

The vast majority of microvascular anastomoses for veins and arteries are performed using hand-sewn sutures. Sutures are tedious, time consuming and technically challenging, even though they provide a reliable, effective anastomosis. In microvascular anastomosis, the veins and arteries range in diameter from two to four millimeters, requiring the use of a microscope. For this reason, microvascular anastomosis has risks, including damage to vessel walls from the stitching and thrombosis (i.e., clot formation in a blood vessel restricting blood flow to portions of the body).

Due to the increasing difficulty of microvascular surgery, the medical community is seeking out more efficient tools and techniques. Current alternative methods to suturing can be divided into five categories: stapling, clipping, gluing, laser welding, and coupling. Stapling techniques have not been widely received by surgeons for a number of reasons. Although this method of anastomosis has proved to be more efficient than hand-sewn suturing, the instrumentation required is cumbersome. Circular staplers are not effective enough to replace suturing due to the fact that they tend to require normal vessels and long sections of eversion for the vessel ends.

Clipping techniques differ from the stapling techniques mainly due to the non-penetrating clips used. The system consists of forceps used to evert the vessel walls and a self-releasing clip applicator that dispenses up to 25 titanium microclips. The YCS is an improvement on traditional stapling methods because like stapling techniques, it is much more efficient than suturing, but the non-penetrating clips also eliminate the need for foreign bodies in the lumen. The drawbacks to this method are that it requires soft, not rigid, vessel walls due to the need for eversion and also requires precise vessel preparation. Studies have shown that it can cause

“have deleterious effects in the early postoperative period” (Keskil et al. (1997) Acta Neurochir (Wien). 139(1):71-6).

The other techniques also have drawbacks. Adhesives are widely used in medical procedures, but are not used as a standalone coupler in anastomoses as they cannot provide the same mechanical joint strength as other methods. Laser techniques have been used experimentally in vascular anastomoses since 1979, but laser welding cannot be used as a standalone technique; it must be combined with the use of a few (four to five) stitches in order to achieve a successful anastomosis. Like clipping techniques, coupling requires eversion of the vessel wall with subsequent joining of the vessel ends with a polyglactin collar.

Therefore, there is a need for devices and methods that provide simpler and more efficient generation of microvascular anastomoses, while providing substantially similar results to known techniques.

### SUMMARY OF THE INVENTION

According to aspects of the present disclosure, methods and devices are disclosed that reduce the time required to perform these microvascular surgeries. In addition, the devices disclosed herein are functional on both arteries and veins. Additionally, the devices and methods disclosed herein are easy to use. More specifically, anastomosis is made simple enough that a highly specialized surgeon is not required to perform the operation. The total amount of time per procedure when using the devices and methods disclosed herein can be reduced to below 10

minutes from the standard 60-120 minutes required now.

Aspects of methods disclosed herein include a method of performing an anastomosis in a microvessel. The method comprises providing a self-expanding stent having one or more members attached thereto to constrain the stent and inserting a first portion of the stent into a first end of a microvessel and a second portion of the stent into a second end of the microvessel. The method further comprises contacting the first end of the microvessel to the second end of the microvessel and deploying the stent by releasing the one or more members from the stent. In addition, the method comprises sealing the first and second ends of the microvessel together.

In certain embodiments, the one or more members is a clasp. In particular embodiments, the one or more members is a biodegradable sheath. In more particular embodiments, the biodegradable sheath comprises bioresorbable materials, polysaccharides or water-soluble polymers.

In certain embodiments, the member comprises water. In other embodiments, the biodegradable sheath comprises sucrose. In some embodiments, the stent has a constrained diameter of less than 3.0 mm or, alternatively, constrained diameters of 1.5 mm. In further embodiments, the stent has a length of less than or equal to 20 mm or a length of 10 mm.

In certain embodiments, the first and second ends of the microvessel are sealed together by tissue glue. In other embodiments, the biodegradable sheath is released from the stent by dissolution of the sheath. In still other embodiments, the biodegradable sheath is released from the stent by melting of the sheath. In some embodiments, the member is released from the stent by the application of a physical force (e.g., pressure).

In some embodiments, the biodegradable sheath is a bioresorbable wrap. In other embodiments, the bioresorbable wrap is released by removing sutures.

In particular embodiments, the stent comprises a material selected from the group consisting of stainless steel, nitinol, polylactic acid, polylactic acid-polybutylene succinate, and cobalt-chromium alloy.

Aspects of couplers are also disclosed herein. In certain aspects, a microvascular anastomotic coupler comprises a stent having at least a portion of a surface of the stent covered by at least one member configured to constrain the stent to a diameter of less than or equal to 3.0 mm.

In particular embodiments, the at least one member is a biodegradable sheath. In other embodiments, the stent comprises a material selected from the group consisting of stainless steel, nitinol, polylactic acid, polylactic acid-polybutylene succinate, and cobalt-chromium alloy.

In particular embodiments, the stent comprises a drug-eluting material. In some embodiments, the stent has a length of less than or equal to 20 mm or, alternatively, the stent has a length of 10 mm. In certain embodiments, the sheath (e.g., biodegradable) is configured to constrain the stent to a diameter of 1.5 mm. In some embodiments, the stent comprises an adhesive material selected from the group consisting of fibrin, cyanoacrylate, and photopolymerizable sealants.

In particular embodiments, the sheath covers (e.g., biodegradable) substantially the entire surface of the stent. In certain embodiments, the biodegradable sheath comprises bioresorbable materials, polysaccharides or water-soluble polymers. In other embodiments, the member or sheath comprises water. In particular embodiments, the biodegradable sheath comprises sucrose.

In more particular embodiments, the biodegradable sheath is molded to the stent. In still more particular embodiments, the member is molded to the stent.

In some embodiments, the biodegradable sheath is a bioresorbable wrap. In other embodiments, the at least one member is a clasp.

Aspects of kits are further disclosed herein. In some aspects, a kit for performing an anastomosis in a microvessel comprises instructions for performing an anastomosis in a microvessel using a stent having one or more members constraining the stent. In still other aspects, the kit further comprises one or more of: i) a stent having a constrained diameter of less than or equal to 3.0 mm; and/or ii) a mold assembly comprising an interior surface defining a space configured to receive the stent.

In certain embodiments, the one or more members is a clasp. In other embodiments, the one or more members is a biodegradable sheath.

In particular embodiments, the kit further comprises instructions for producing a sheath (e.g., biodegradable) utilizing the mold assembly. In more particular embodiments, the kit further comprises one or more materials selected from the group consisting of bioresorbable materials, polysaccharides, water-soluble polymers, and water. In other embodiments, the one or more materials are sucrose or water.

In still other embodiments, the stent comprises a material selected from the group consisting of stainless steel, nitinol, polylactic acid, polylactic acid-polybutylene succinate, and cobalt-chromium alloy. In further embodiments, the stent has a length of 10 mm. In still further embodiments, the stent has a constrained diameter of 1.5 mm.

In some embodiments, the kit further comprises a tissue glue. In still more embodiments, the stent comprises an adhesive material selected from the group consisting of fibrin, cyanoacrylate, and photopolymerizable sealants.

Aspects disclosed herein further disclose methods of making an anastomotic coupler. In certain aspects, the method comprises providing a mold assembly, placing a material and a stent into the mold assembly, and producing an anastomotic coupler comprising the stent surrounded over at least a portion of its surface by one or more sheaths.

In certain embodiments, the one or more sheaths comprise bioresorbable materials, polysaccharides, water-soluble polymers, or water. In other embodiments, the one or more sheaths comprise water or sucrose.

In still other embodiments, the stent has a constrained diameter of less than 3.0 mm. In additional embodiments, the stent has a constrained diameter of 1.5 mm. In further embodiments, the stent has a length of less than or equal to 20 mm or, alternatively, the stent has a length of 10 mm. In still further embodiments, the stent comprises a material selected from the group consisting of stainless steel, nitinol, polylactic acid, polylactic acid-polybutylene succinate, and cobalt-chromium alloy.

### DESCRIPTION OF THE FIGURES

The following figures are presented for the purpose of illustration only, and are not intended to be limiting:

FIG. 1 is an illustration of an anastomotic coupler comprising a sheath surrounding and constraining a stent.

FIG. 2 is an illustration of a biodegradable sheath that is a bioresorbable wrap.

FIG. 3A is a side view of a stent constrained by a clamp having a latch mechanism that is detachable by applying pressure to the stent.

FIG. 3B is a cross-sectional view of the stent in FIG. 3A.

FIG. 3C is a side view of an alternative clamp mechanism.

FIG. 3D is a cross-sectional view of the clamp mechanism of FIG. 3C.

FIG. 4 is an illustration of a sheath that is an alternative embodiment of a clamp.

FIG. 5 is an illustration of a sheath that is composed of ice.

FIG. 6 is an illustration of an embodiment of a mold assembly for producing a sheath.

FIG. 7 is an illustration of a sheath produced using the disclosed mold assembly.

FIG. 8 shows the temperature change plotted against time of temperature measurements performed on ice sheaths.

FIG. 9 shows the results of expansion of the stent as the ice sheath was melted during temperature tests. A: The sheath is fully formed at the top of the figure and fully constrains the stent. B: When the sheath begins to dissolve/melt, the stent is released and partially expands, as shown in the middle. C: The stent shown at the bottom of the figure is completely released due to the sheath being dissolved/melted.

FIG. 10 shows the results of a body temperature melt test performed on a stent to determine the behavior of the stent as the ice sheath melted. A: A sheath is shown constraining the stent. B: One side of the stent is released when one half of the sheath melts. C: The entire stent is released when the entire sheath melts.

FIG. 11 is a sample P-h curve of data from a force displacement indentation test, where P is the indentation force and h is the depth.

FIG. 12 is a photograph showing a stent being constrained by a sheath.

### DETAILED DESCRIPTION OF THE INVENTION

### 1. General

The patent and scientific literature referred to herein establishes knowledge that is available to those of skill in the art. The issued US patents, allowed applications, published foreign applications, and references that are cited herein are hereby incorporated by reference to the same extent as if each was specifically and individually indicated to be incorporated by reference.

According to aspects of the present disclosure, a microvascular anastomotic coupler is disclosed. The coupler comprises a stent having a portion of a surface of the stent covered by at least one member configured to constrain the stent. In some embodiments, all or substantially all of the stent is covered by the at least one member. In certain embodiments, the stent is self-expanding.

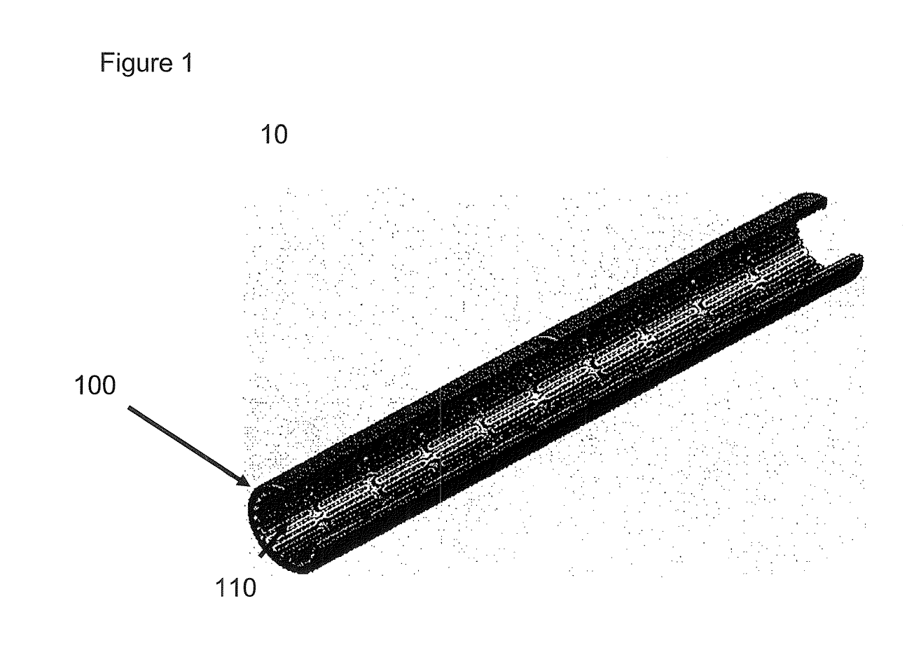

An exemplary embodiment of an anastomotic coupler is shown in FIG. 1. The coupler 10 comprises a stent 110 and a single member 100 constraining substantially all of the stent 110. As is clear, the member constrains the stent 110 by applying force to the outside of the stent 110. In this embodiment, the member 100 completely surrounds the stent 110 to maintain the stent 110 in its constrained state. It should be understood that one or more members can be used to constrain the stent or simply one member can be used to constrain substantially all of the stent.

Stents can be small, mesh wire devices that are used as scaffolding in blood vessels to prop the vessel open. Once in the appropriate location, the stent is deployed by expanding the stent. After the stent is deployed, increased blood flow through constricted areas results from the minimally invasive procedure. For typical procedures, the stents need only be deployed to maintain vascular patency for a period of approximately four weeks, as a collateral network of blood vessels will have formed in grafting procedures by that time. While the graft may have full blood supply after a shorter amount of time, it is necessary to include a safety factor to account for any complications.

Returning to FIG. 1, stent 110 can be a multitude of designs (for example, self-expanding designs) including bare-metal, drug-eluting, polymer, and biodegradable/bioresorbable polymer. In certain embodiments, the stent of the coupler is composed of stainless steel, nitinol, polylactic acid, polylactic acid-polybutylene succinate, and cobalt-chromium alloy. Furthermore, the stents can be coated with drugs such as a paclitaxel, rapamycin, hirudin, iloprost, GPIIb/IIIa inhibitors, angiopeptin, somatostatin, tyrosine kinase inhibitors, methylprednisolone, and prostacyclin. Such coatings are well known in the art (see, e.g., Gunn and Cumberland, (1999) European Heart Journal 20:1693-1700). Additionally, the stents can be passively coated using techniques known in the art. As used herein, the term “passive coating” refers to coatings that provide a barrier between the stent surface and a tissue such as the endothelial wall of a blood vessel and blood. Exemplary passive coatings include gold, heparin, carbon. silicon carbide, polylactic acid, organophosphazene, polyurethane, titanium-nitride-oxide, fibrin, and phorphorylcholine. In addition, the stent can have markers that allow tracking and identification of the stent. Such markers include tantalum and other radio-opaque markers.

In certain embodiments, the microvascular anastomotic coupler is useable on both veins and arteries of 2-4 mm diameters. The stents can have constrained diameters of less than or equal to 3.0 mm. In particular embodiments, the stents have a constrained diameter of less than 1.5 mm. In its deployed state, the stents can have a diameter that is about 15-20% larger than the diameter of the unstrained vessel. In additional embodiments, the stent is short enough in the axial dimension to be manipulated in a microsurgery environment, while still being long enough to provide the necessary surface contact area on the vessel lumen to prevent the coupled vessel from separating axially.

In addition, the stent 110 of the coupler 10 in FIG. 1 has a range of lengths that are effective for use in anastomosis generation. Benchmark testing show that this length can be approximately 10 mm. In certain embodiments, the stent has a length of less than or equal to 20 mm.

The stent of the coupler can include an adhesive for the coaptation of vessel ends to aid in the withstanding of diastolic and systolic blood pressures and ensure a leak-free anastomosis. The adhesive can be biocompatible to avoid harm to the body including thrombosis, aneurysm, or toxicity. Commonly used adhesives for such applications include fibrin, cyanoacrylate, and photopolymerizable sealants.

There are several stent fabrication methods including coiling, braiding and knitting of wire, laser cutting of tubing, and photochemical etching. These fabrication methods are used to produce a multitude of stent geometries that have varying mechanical properties. Coil geometries allow for stent retrieval after implantation, however, they have limited strength and a low expansion ratio. Helical spiral configurations have the advantage of being flexible, but are less stable longitudinally; by adding internal connection points, longitudinal stability is gained at the cost of some flexibility. Woven-braided stent designs are often used for self-expanding structures that offer excellent coverage, but shorten substantially during expansion. In these structures the radial strength is highly dependent on axial fixation of its ends. Sequential ring geometries compose the majority of vascular stent designs because of the many complex geometries they can provide: open cell, closed cell, regular connection, periodic connection, and peak-peak or peak-valley connections can all be utilized depending on the requirements of the stent application. The fabrication techniques discussed herein are known (see, e.g., Stoeckel et al. (2002) Min Invas Ther & Allied Technol 11(4): 137-147). Additionally, stents are available commercially from, for example, Abbot Vascular (Santa Clara, Calif.).

Returning to FIG. 1, the member 100 can be any material that releases the stent from its constrained state. In certain embodiments, the member is a biodegradable sheath. Biodegradable sheaths disclosed herein can be a multitude of designs, including bioresorbable caps, bioresorbable wraps, clamps, and external hooks. The biodegradable sheaths can be made from any bioresorbable material, such as polysaccharides, biocompatible salts (e.g., NaCl), poly-L-lactide, or frozen water. The biodegradable materials should be sterile to prevent infection. Additionally, biodegradable sheaths can comprise additional biocompatible materials such PEG. Furthermore, biodegradable sheaths can comprise polysaccharides obtained from natural sources such as honey, sugar cane, refined sugar, and molasses.

In certain embodiments, a bioresorbable member constrains substantially all or all of a collapsed stent, thereby preventing the stent from expanding. These members could either be a sheath that extends to full length. The surgeon can place the first end of the coupler into the first end of a vessel in its correct orientation. The surgeon then applies pressure to the outside of the vessel wall, breaking the sheath and releasing that portion of the stent. The process is repeated for the other end of a vessel. The two vessels, or two ends of the same vessel, are then secured in the middle with adhesive. The sheath is absorbed by the vessel walls as healing occurs.

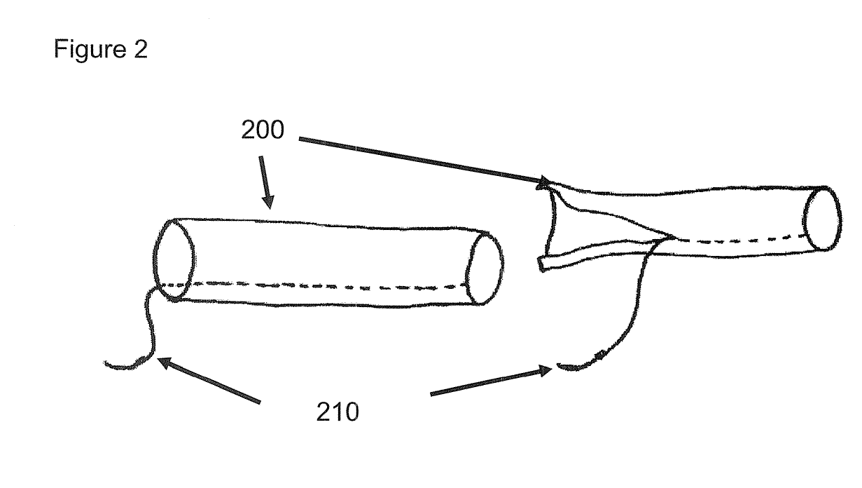

As displayed in FIG. 2, a bioresorbable wrap 200 design collapses the stent using a sheath around the outside of the stent to constrain it. Sutures 210 are woven into the wrap, and in the same manner as the previous suture design, pierce through the vessel wall. As the surgeon pulls the suture, the wrap releases on one side of the stent and it expands. In the same manner, the other side of the stent is released, and the vessels, or two ends of the same vessel, are sealed together using adhesive.

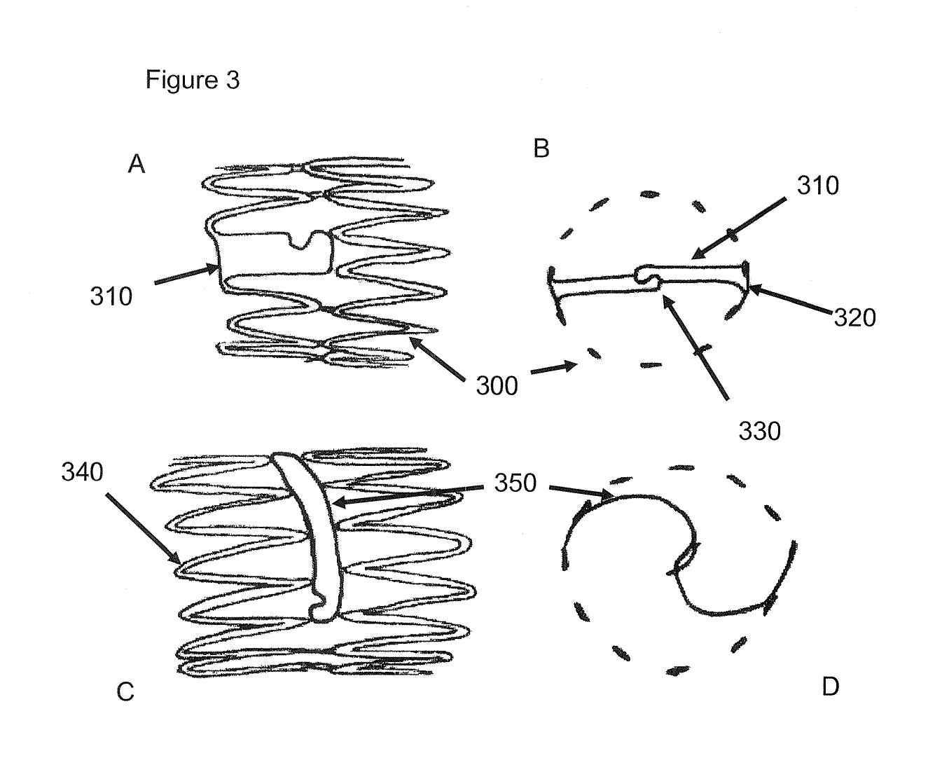

As shown in FIG. 3A, a clamp 310 integrated within the weave of the stent 300 constrains the stent 300 of the coupler. A portion of the weave 320 of the stent 300 can be designed into hooks with one pair of hooks 330. With the stent 300 collapsed, these hooks 330 would bend towards the central axis of the stent, catching each other and holding the stent in the collapsed position. The surgeon would place one end of the stent into a vessel, and apply force to the collapsed stent. This force can be as simple as squeezing the end of the stent 300 to apply pressure to release the hooks 330. This force would cause the hooks 330 to unlatch, allowing the stent 300 to expand. The hooks 330 can be made of the same material of the stent. For instance, the hooks would be able to return to the at rest state in line with the stent wall, so as to not induce turbulence in the blood flow. This process would be repeated for the other vessel end. The hooks can be arranged in either an axial or circumferential manner. An alternative embodiment is shown in FIGS. 3C and 3D in which the clamp 350 is formed along the circumference of the stent 340.

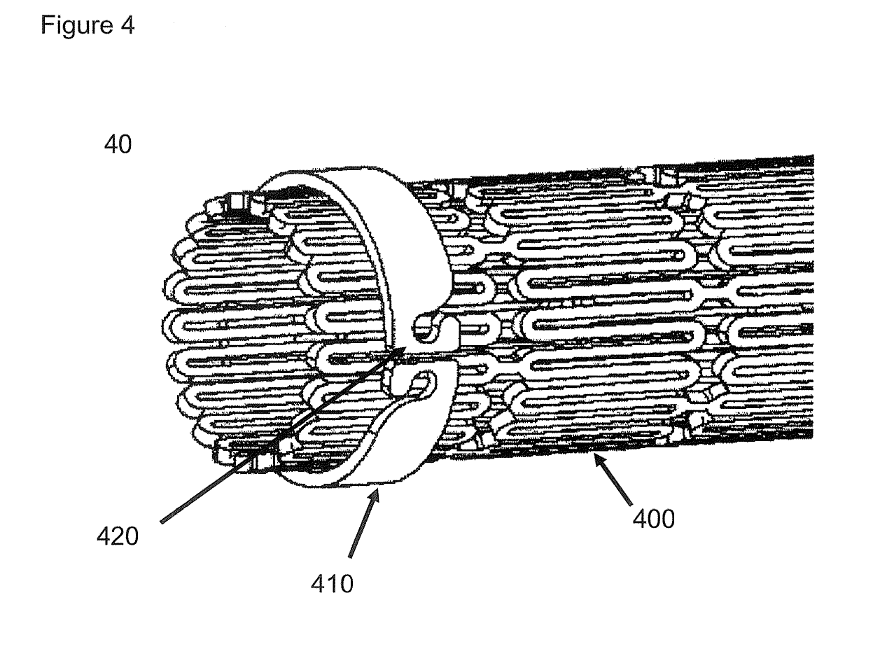

FIG. 4 shows an anastomotic coupler 40 comprising a stent 40 constrained by a member 410 having an external circumferential hook design, where the hooks 420 are not integrated into the weave itself, but are attached either through welding or crimping. FIG. 4 shows that this design utilizes circumferential hooks 420 that engage on the exterior of the stent 400, negating all possible turbulent effects due to hooks within the stent. The hooks 420 can be attached to the stent 400 at one point, a point on the opposite side of where the hooks 420 latch together. The external hooks 420 can be made such that when the stent is collapsed, the hooks are able to be forced to catch. When the operator applies force (i.e., pressure) to the member 410, the stent 400 expands and the hooks 420 disengage and match the diameter of the stent 400. Although FIG. 4 shows hooks constraining an end portion of the stent, it should be noted that the hooks 410 should constrain substantially all or all of the stent 400.

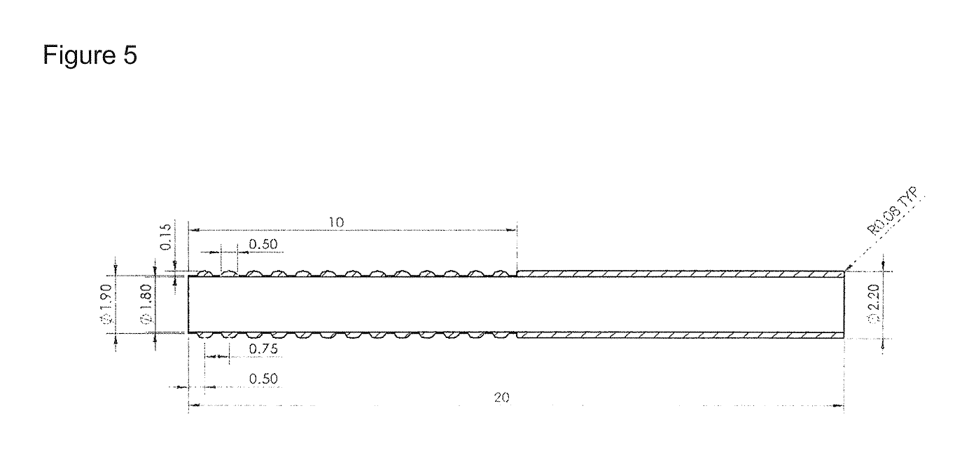

In a particular embodiment, a member is an ice sheath that is smaller in diameter than the vessel to allow proper placement. In such embodiments, the ice sheath is a biodegradable sheath that melts to release the stent. In this embodiment, one half of the length of the ice sheath is smaller in diameter and mass than the other half. This smaller half also features raised ribs to increase surface area (FIG. 5). The design biases one side (the smaller, ribbed side) of the sheath to a faster melting time. In use, the smaller side is inserted into one vessel and remains there until melted or fractured by the surgeon. Once the ice is melted or the sheath ruptures, the stent will deploy. Then the other side is inserted into the second vessel end and the process is repeated. An alternative embodiment is to cover the second end in an insulating wrap. This relieves the time constraint the surgeon must work with in order to prevent the second end from deploying prematurely. In addition to the outer sheath, the stent can have a supportive core of ice to aid in both the manufacture of the design and to stabilize the temperature of the assembly.

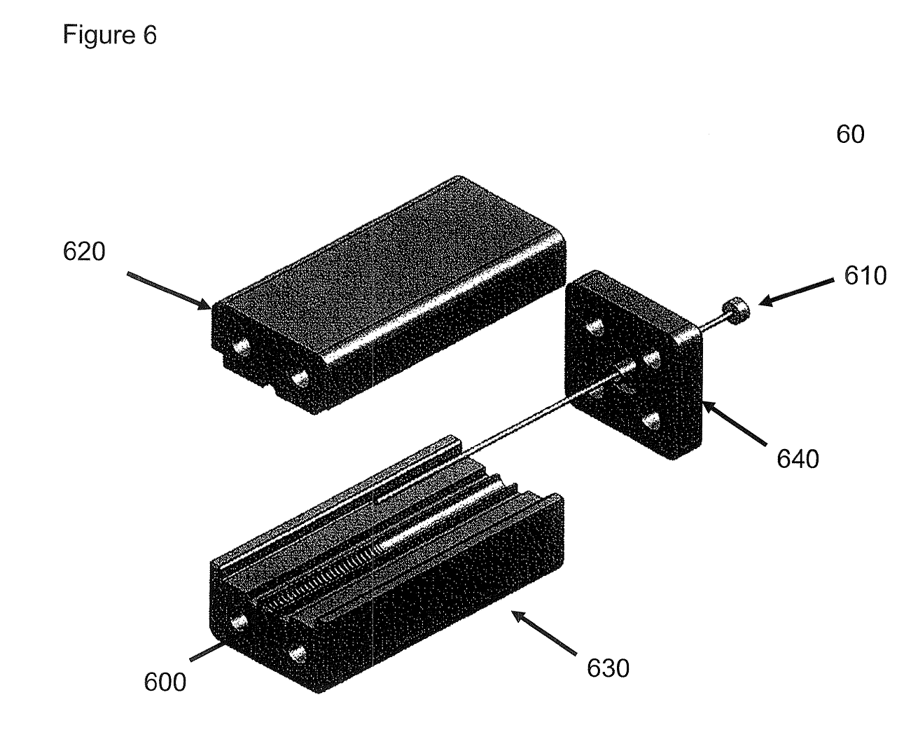

The ice sheath design can be produced using a mold. The mold can be dimensioned based on the measurements of stents. In one embodiment, the mold consists of a three-part mold that to manufacture the ice sheath. In another embodiment, the second assembly modeled the ice sheath and supportive core. FIG. 6 shows an exploded view of an exemplary mold 60. The mold 60 comprises a top portion 620, side portion 640, and a bottom portion 630. The mold also comprises a pin 610 that is useful for extruding the anastomotic coupler after it has been made. The mold 60 can also has a mold set 600 that allows for sheaths of particular dimensions, thicknesses, and patterns to be produced.

In certain embodiments, water, polysaccharide solutions, or biodegradable polymers are placed into the mold set 600 of fully constructed mold 60. A stent is then placed into the water, polysaccharide solution, or biodegradable polymer in mold set 600. The water, polysaccharide solutions, or biodegradable polymers are allowed to set either by freezing or any other polymerization reaction known to those of skill in the art. The pin 610 is used to extrude the anastomotic coupler from the mold set 600.

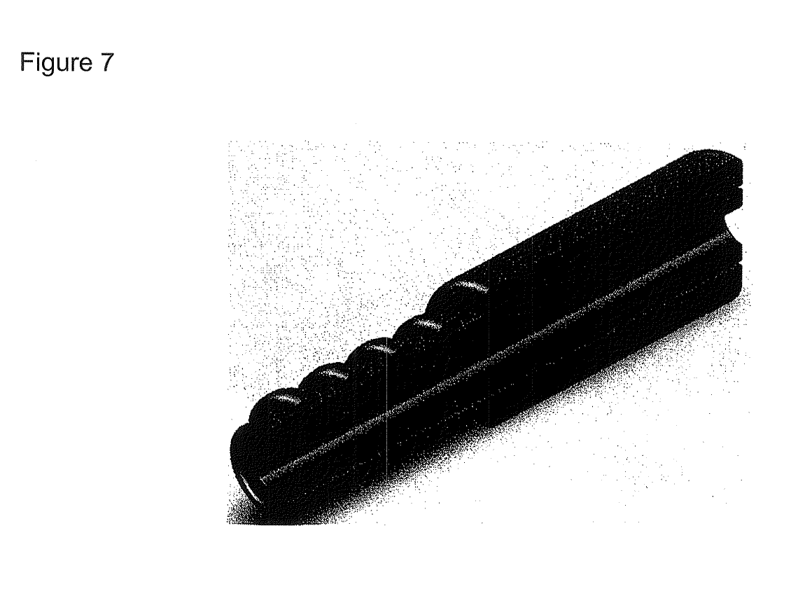

FIG. 7 shows a section view of the ice sheath formed by the mold. In this embodiment, the ice sheath has an inner diameter of 2.13 mm, the ribs have a radial thickness of 0.15 mm and width of 0.5 mm, and the larger section has a radial thickness of 0.15 mm.

Aspects disclosed herein include methods of performing an anastomosis in a microvessel. The methods comprise providing a coupler comprising a stent having one or more biodegradable sheaths constraining the stent. The methods also comprise inserting a first portion of the stent into a first end of a microvessel and a second portion of the stent into a second end of the microvessel. The first of the microvessel is contacted to the first end of the microvessel to the second end of the microvessel. The stent is deployed by permitting the one or more sheaths to be released from the stent. In certain embodiments, the method of forming an anastomosis includes sealing the first and second ends of the microvessel together.

The coupler can be placed into the site for forming an anastomosis using forceps. In certain embodiments, the coupler is placed using reverse-action tweezers. The reverse-action tweezers have cylindrical tip shapes. These cylindrically shaped tips allow the surgeon to easily and reliably hold and maneuver the ice sheath. The reverse-action feature also can increase the ease of use by requiring activation force only when the surgeon plans to release the device. This feature prevents the likelihood of dropping or misplacing the sheath during the operation. The tip of the tweezers can be coated or replaced with an insulating material such as plastic or rubber to further protect the ice sheath and prevent unintentional melting or dissolution of the biodegradable sheath.

The coupler can be deployed such that the stent can be expanded from the outside of the vessel while the device is entirely within the vessel. This deployment can be local, within the surgical site surrounding the vessel. Additionally, the stent can be deployable on either side independently, i.e., only one side need be deployed at a time. This characteristic deployment should not interfere with the stresses exerted by the stent.

Aspects disclosed herein include kits for performing an anastomosis in a microvessel. The kits comprise instructions for performing for performing an anastomosis in a microvessel using a stent having one or more biodegradable sheaths constraining the stent. In certain embodiments, the kits include written instructions or instructions that are provided on the internet. Such instructions provide protocols on how to use the couplers disclosed herein. For instance, the instructions can provide protocols for performing an anastomosis in a microvessel. In addition, the instructions can explain how to make the sheaths that surround at least a portion of the stent.

In certain embodiments, the kits additionally comprise a stent having a constrained diameter of less than or equal to 3.0 mm. In certain other embodiments, the kits comprise a mold assembly comprising an interior surface defining a space configured to receive the stent. In some embodiments, the kits comprise a stent and a mold assembly.

Furthermore, the disclosed kits can provide the components to make a biodegradable sheath. For instance, one or more biodegradable materials can be provided. The materials include bioresorbable materials, polysaccharides, such as sucrose, water-soluble polymers, poly-L-lactide, and water. The kits can also include a stent that comprises a material selected from the group consisting of stainless steel, nitinol, polylactic acid, polylactic acid-polybutylene succinate, and cobalt-chromium alloy. In other embodiments, the stent has a length of 10 mm and/or a constrained diameter of 1.5 mm.

In certain aspects, the kits solely comprise a mold for making a biodegradable sheath. In other aspects, the kits further comprise a pre-fabricated biodegradable sheath, such as a wrap, clamp, or sutures to secure the stent. In other embodiments, the kit includes a tissue glue. In kits containing a stent, the stent comprises an adhesive material selected from the group consisting of fibrin, cyanoacrylate, and photopolymerizable sealants.

### 2. Experimental

An embodiment of the ice sheath was imported into Abaqus to undergo finite element analysis, both for heat transfer and stress. To model the ice sheath, the data in Table 1 were used.

TABLE 1

| Property | Value | Unit |
|---|---|---|
| Young's Modulus | 9 | MPa |
| Tensile Strength | 8.5 | MPa |
| Poisson's Ratio | 0.33 | n/a |
| Density | 916.7 | kg/m³ |
| Conductivity | 2.2 | W/mK |
| Specific heat | 1700 | J/kgK |
| Latent Heat | 334 | kJ/kg |

In addition to the properties of ice, the thermal conductivity of human blood vessels was needed to perform a heat transfer analysis. This value is 0.464 W/mK for the human aorta, and similar values are found for different arteries of other animals (Marcus et al. (1981) Circ. Res., 48: 748-761). Using the thermal conductivity and table values, the heat flux applied to the ice was found to be −17.168 W/m². In order to increase the resolution of the model, only one quarter of the ice was imported. The imported model simulated the ice sheath with the supportive core, and assumed a stent thickness of 0 mm to consider the ice enveloping the stent. The results from this analysis can be extrapolated to the whole due to the axis symmetry of the design. The entire sheath and core assembly was set to 0° C. at the start of the analysis. The results establish that the time to melting of the sheath is relatively short (minutes), but not so short that the surgeon needs to work too quickly.

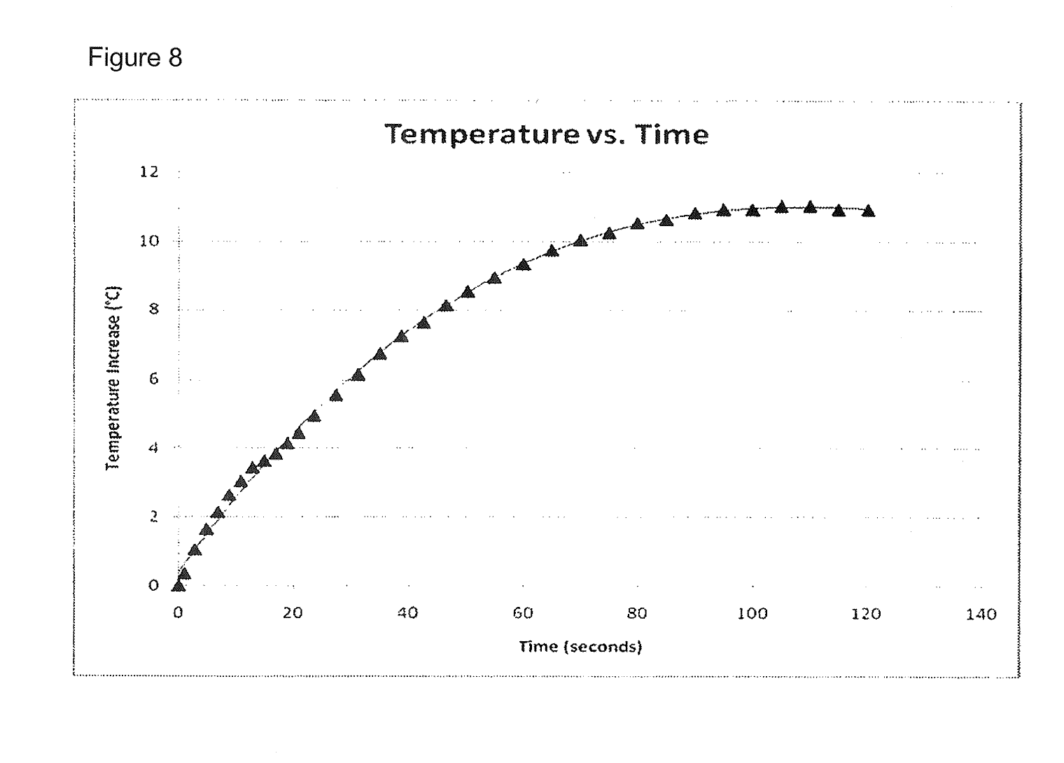

In addition to the heat flux data, temperature measurements were made to further quantify the thermal behavior of the coupler. The ice sheath behaved as a solid body with respect to temperature, meaning that the temperature was consistent throughout the entire sample. As such, FIG. 8 shows the temperature change plotted against time. The graph shows that after 30 seconds the temperature has increased by approximately 5° C., and by the end of 120 seconds the ice has warmed up by approximately 11° C. The deviations of the data from the fit line can be explained by the resolution of the elements of the model, which are limited to 20,000 by the student edition of the software. Additionally, this model does not consider the phase change of the ice, which undoubtedly affects the heat transfer. This discrepancy was instead tested using an experimental model.

The ice sheath was also modeled, using the same properties from Table 2, to determine if it could withstand the pressures exerted by the constrained stent. When deployed, the stent should exert around 100 kPa on the interior surface of the blood vessel. The ice sheath was subjected to this 100 kPa outwards pressure on the inner surface. Additionally, the inner supportive core was removed from this model to accurately depict the behavior of the system. The maximum principal stress in the simulation is approximately 1.94 MPa, approximately 4.5 times lower than the tensile strength of ice (−8.5 MPa). This analysis, however, does not combine the melting of the sheath with its strength, so bench testing is required to verify its ability to not crack under pressure. If both the stress and heat transfer data are proven to be accurate, then the sheath is currently overdesigned and can be thinned out.

As a preliminary test to prove the mold functions as designed, the mold was lubricated with a silicone grease mold release, one end of the mold was capped, and the whole mold was filled with water using a needle and syringe. The assembly was placed in a typical household freezer, and left for 2 hours. After 2 hours, the mold was disassembled and the ice was removed. This process was repeated two more times. Depending on the coverage of the mold release, the ice would remain either fully intact or break into multiple pieces. The ice pieces would melt between 30 to 60 seconds, depending upon the size of the fragments, in an environment ranging from approximately 15° C.-18° C. An intact piece would melt in close to or approximately 60 seconds, while a specimen that fractured in more than three pieces would melt in closer to 30 seconds.

Once it was proven that the mold would produce ice in the designed geometry, the ice sheath's ability to constrain was tested. The mold was prepared as in the previous tests, with a 6 mm diameter, 38 mm long stent (DynaLink 0.018 Biliary Self-Expanding Stent System) partially deployed into the water-filled mold. Because the stent was 38 mm long, and the delivery mechanism was designed for an open tube, instead of a capped one, approximately 1 cm of the stent remained outside of the mold. Water was then added, and the assembly was placed in the freezer overnight. Once removed from the freezer, the constrained portion of the stent remained fully constrained for 60 seconds until the ice melted sufficiently for expansion of the stent.

Mold adjustments included the removal of the ribs and an increase of 0.25 mm in the thickness of the ice cylinder. Once this new mold was created, the following procedure was used for testing: the mold and pin were greased and assembled, water was added and then the entire assembly was placed in a −80 oe freezer for 5-7 minutes, the pin was taken out and a stent was deployed into the outer ice sheath, a solid end cap was attached to the mold, 0° C. water was added, the assembly was placed back into the −80° C. freezer for an additional 5-7 minutes, the assembly was removed for testing.

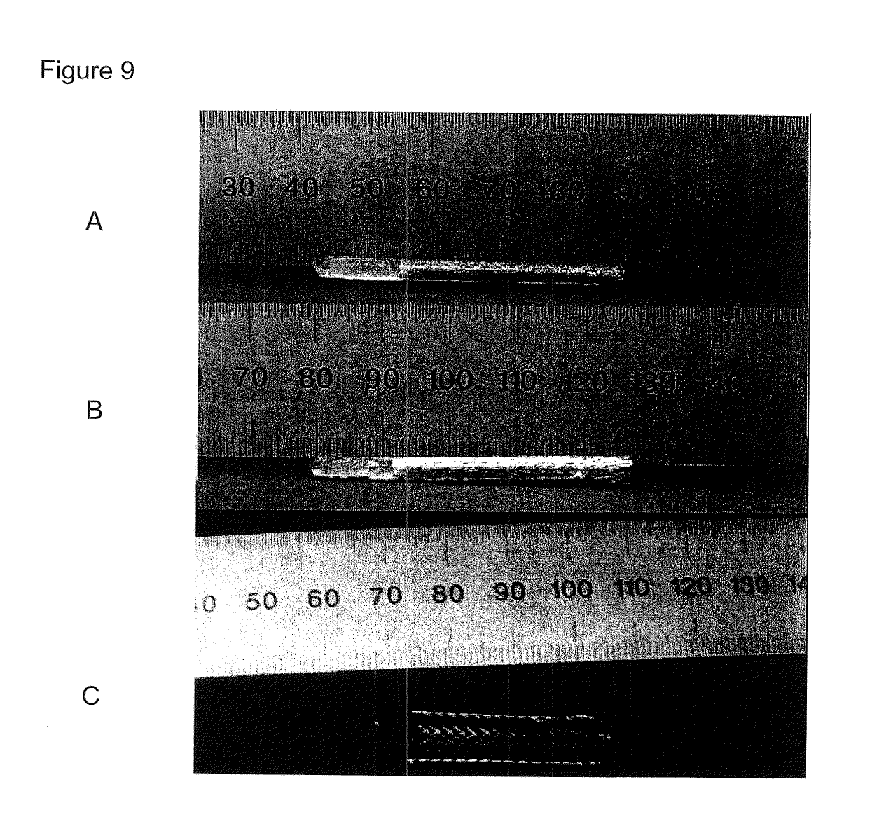

The room temperature melt test entailed removing the stent assembly from the mold, placing it on a piece of lightweight foam, and noting the time it took for the stent to fully expand. The stent used in the present test was a 9 mm×30 mm, RX Acculink carotid stent system (Abbott Vascular). The stent began to expand at around 8 minutes, but did not achieve full expansion until 11 minutes. FIG. 9 shows the behavior of the stent throughout the test. In particular, FIG. 9A shows that the stent remained in a constrained state when surrounded by the biodegradable sheath, in this case, an ice sheath. In FIG. 9B, the sheath begins to melt and the stent becomes less constrained. FIG. 9C shows that the stent is now completely unconstrained due to the melting of the ice sheath.

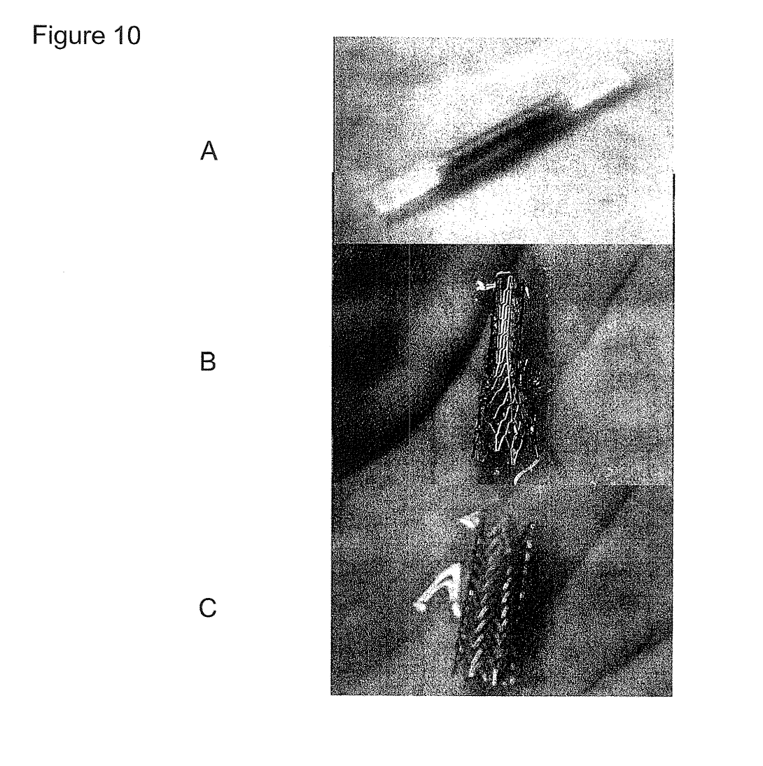

A body temperature melt test was performed in a manner similar to the room temperature melt, except that the stent (7 mm×20 mm, RX Acculink carotid stent system, Abbott Vascular) was placed on a human hand instead of a piece of foam. Expectedly, the stent melted much faster at this temperature: partial expansion occurred after just 75 seconds, while full expansion was complete by 105 seconds. FIG. 10 shows this process. FIG. 10A shows a fully constrained stent surrounded by an ice sheath. FIG. 10B shows one end of the stent expanding as the ice sheath melts. FIG. 10C shows a completely expanding stent due to the melting of the ice sheath.

The last tests performed involved creating the sheath and stent assembly, and then using it to join two vessel analogues. The first mock anastomosis procedure went well, with the sheath melting and the stent expanding into the vessels. Additionally, the mock surgeon was able to crush the sheath from outside the vessel wall, aiding in the melting. In certain embodiments, the surgeon may need to deliver the anastomotic coupler using a tool such as forceps, which would allow the surgeon to properly place the sheath before breaking it. The stents used in the mock surgery were a 4 mm×20 mm Abbott Xpert biliary stent system (Abbott Vascular) and a 7 mm×40 mm Guidant ABSOLUTE 0.035 self-expanding stent system (Boston Scientific Corp.).

As a possible alternative material, sucrose (representative polysaccharide) was also tested for its feasibility in constraining a stent. Like the ice sheath, the sucrose sheath is smaller than diameter than the vessel to allow proper placement. Because the sucrose is not as sensitive to temperature, the sheath is a uniform cylinder. One half of the sucrose sheath is inserted into one vessel and is then fractured by the surgeon, deploying half of the stent. Then the other side is inserted into the remaining vessel and is again fractured. The remaining sucrose fragments are dissolved by flushing the vessel out with saline.

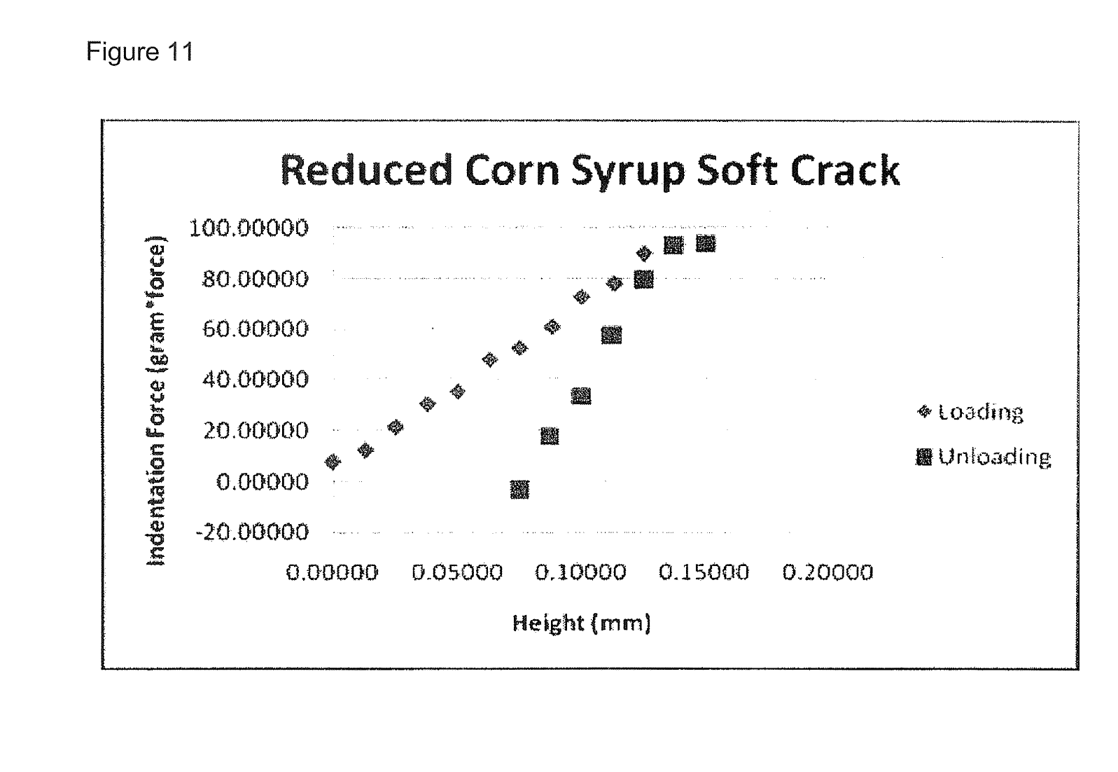

To ensure a thorough study, several different sugar based mixtures were made using only biocompatible materials in addition to the sugar: water, dextrose (corn syrup), and polyethylene glycol (20,000 g/mol). In addition to varying the concentrations of each material, the temperature the mixtures were brought to during preparation was also adjusted. These temperatures are referred to by their common baking terminology: hard crack (approximately 150° C.) and soft crack (approximately 135° C.). Once the mixtures were prepared, they were tested using a force displacement indentation test. The data from these tests were used to create P-h curves, where P is the indentation force and h is the depth. A sample curve can be found below in FIG. 11.

Sugar, in a humid environment, tends to become sticky. This stickiness causes problems during an indentation test, as can be seen from the unloading data points in FIG. 11. The first two points of the unloading curve can be seen as decreasing in depth, while the force remains relatively the same which is a strong indicator of the indenter sticking to the sample. Despite the sample sticking during unloading, the loading curves of the samples were consistent enough with typical P-h curves to provide useful data. The resulting data were used to calculate the Young's modulus of the sugar blends, using the Hertzian Equation 3, where P is the load, r is the radius of the indenter (4 mm from testing), E is the desired Young's modulus, v is the Poisson's ratio of the sample (estimated to be 0.4), and h is the depth:

$$P = 4 / 3 \cdot {r}^{1 / 2} ( \frac{E}{1 - {v}^{2}} ) {h}^{3 / 2} \qquad ( 1 )$$

These calculated modules, along with additional information about the sugar samples, can be seen below in Table 2.

TABLE 2

| | | | Modulus | |
|---|---|---|---|---|
| Description | Composition | Temperature | (average, Mpa) | # Tests |
| Hard Crack | 1 cup sugar, 1/2 cup water, ⅓ | 150-155° C. | 4.80 | 4 |
| | cup corn syrup | | | |
| Soft Crack | 1 cup sugar, 1/2 cup water, ⅓ | 135-140° C. | 8.63 | 3 |
| | cup corn syrup | | | |
| Reduced Corn Syrup | 1 cup sugar, 1/2 cup water, ⅙ | 135-140° C. | 15.67 | 9 |
| Soft Crack | cup corn syrup | | | |
| Pure Sugar | 1 cup sugar | 160-165° C. | 3.38 | 10 |
| Sugar and Water | 1 cup sugar, 1/2 cup water | 135-140° C. | 28.67 | 10 |
| Sugar, Water, and | 1 cup sugar, 1/2 cup water, 15 mL | 135-140° C. | 17.16 | 10 |
| PEG | PEG | | | |

The modulus of sugar varies according to imperfections in the mixture, caused by bubbles or imperfect dissolution. In the case of the sugar and water mixtures, indentations made near the center of the sample had an average modulus of about 17 MPa, while tests performed on the edge of the sample had an average of about 41 MPa. As such, while the combined average modulus of a sugar and water mixture is about 29 MPa, it is safer for design purposes to assume the lower modulus value to be around 17 MPa. Similarly, the average modulus for sugar, water, and polyethylene glycol (PEG) is around 17 MPa, but tests performed near the center of the sample averaged around 34 MPa, and tests taken on the edge of the sample averaged 0.43 MPa. The significant difference in the mixtures composed of sugar, water, and PEG can likely be attributed to incomplete dissolution of the PEG during the manufacture of the sample. In addition to the modulus testing, several three point beam fracture tests were performed using the sugar and water mixture. The average yield stress of this sugar blend is 0.19 MPa. Thus, the modulus of sugar will be taken as 17 MPa with a yield stress of 0.19 MPa for all following calculations. To determine the minimum sheath thickness required to constrain a stent, calculations were made using Laplace's law concerning pressure in a cylinder:

$$t = \frac{P \cdot r}{σ} \qquad ( 2 )$$

Where t is the tube thickness, P is the internal pressure, r is the inner radius of the tube, and σ is the stress. Using values of 100 kPa, 1.07 mm, and 0.19 MPa for P, r, and σ respectively, the required thickness of a sugar tube to constrain a stent would be 0.55 mm. This thickness is relatively large when compared to the stents the tube will constrain: a 2 mm diameter stent would have to be collapsed to below 0.9 mm for the assembly to even fit within the blood vessel. While this thickness seems prohibitive, it may be possible to achieve a more stable mixture of sugar, water, and PEG that would lead to both a higher elastic modulus and a higher yield stress.

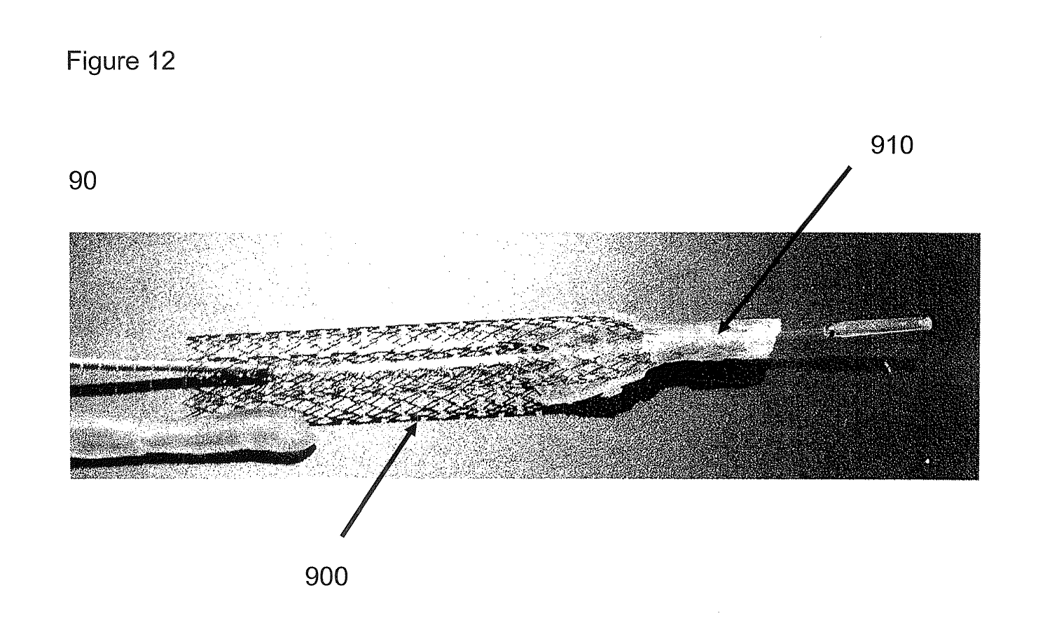

Because the mold for the ice was made with SLA, it was undesirable to use it to test molten sugar. Additionally, because there is no need for heat transfer, the ribbed design for the ice sheath is unnecessary for a sucrose sheath. As such, tubes were made from the sugar mixtures not by a mold process, but by allowing the blends to cool slightly, then stretching the material around a pin and molding by hand. The results achieved by this method are not meant to simulate a fully functioning mold, but instead serve as a proof of concept. Because human manipulation is required, some imperfections in the tubes are expected. A tube made from the Soft Crack mixture of Table 2 was tested using an 8 mm diameter, 60 mm long stent (Abbott Vascular Xceed biliary stent). The tube had an inner diameter of approximately 2.15 mm, meaning it was designed for a stent between 3 and 5 mm in expanded diameter. The tube was also only around 20 mm long, making it significantly undersized for the stent used. Expectedly, part of the tube fracture when the stent was deployed; however, there were preexisting cracks from manufacture in the fractured portion. In the relatively well formed portion of the tube, the stent was fully constrained, as can be seen in FIG. 12. FIG. 12 shows an anastomotic coupler 90 having a sheath 910 that is constraining one end of a stent 900.

Based on this proof of concept, a stent could be safely and fully constrained using a biocompatible mixture based on sucrose. To further test the strength of sugar tubes, samples were made from both the sugar, water, and PEG mixture and the sugar/water mixture. The members (i.e., tubes) had an average wall thickness of 0.21 mm (sucrose+water+PEG), 0.29 mm (sucrose+water+PEG), and 0.55 mm (sucrose+water) in the tests. The stents used in the tests were either the Abbott Vascular 7 mm×100 mm Xceed Biliary stent system or the Abbott Vascular 7 mm×120 mm Xceed Biliary stent system.

Both PEG tubes tested suffered brittle fracture, with the tube in the first test completely shattering after a period of less than 10 minutes. The second PEG tube fractured on both ends, where the stent was exposed, but the center did not break. The tube composed of sugar and water did not fracture at all. This is likely due to both the material properties from Table 2 and the wall thickness of the tubes. The average thickness of the sugar and water tube (0.55 mm) is close to the calculated minimum value from 0.55 mm. It is important to note that testing was performed with oversized stents in these tube deployments because the use of a larger stent than the one designed for increases the pressure acting on the sheath, thereby increasing the likelihood of failure.

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of performing an anastomosis in a microvessel, the method comprising:
a) providing a self-expanding stent having one or more members attached thereto to constrain the stent;
b) inserting a first portion of the stent into a first end of a microvessel and a second portion of the stent into a second end of the microvessel;
c) contacting the first end of the microvessel to the second end of the microvessel;
d) deploying the stent by releasing the one or more members from the stent; and
e) sealing the first and second ends of the microvessel together.

2-10. (canceled)

11. The method of claim 1, wherein the first and second ends of the microvessel are sealed together by tissue glue.

12. The method of claim 1, wherein the at least one member is a biodegradable sheath and wherein the biodegradable sheath is released from the stent by dissolution or melting of the sheath.

13. (canceled)

14. The method of claim 1, wherein the member is released from the stent by the application of a physical force.

15-17. (canceled)

18. A microvascular anastomotic coupler comprising a stent having a surface of the stent covered by at least one member configured to constrain the stent to a diameter of less than or equal to 3.0 mm.

19. The microvascular anastomotic coupler of claim 18, wherein the at least one member is a biodegradable sheath.

20. The microvascular anastomotic coupler of claim 18, wherein the stent comprises a material selected from the group consisting of stainless steel, nitinol, polylactic acid, polylactic acid-polybutylene succinate, and cobalt-chromium alloy.

21. The microvascular anastomotic coupler of claim 18, wherein the stent comprises a drug-eluting material.

22. The microvascular anastomotic coupler of claim 18, wherein the stent has a length of less than or equal to 20 mm.

23. (canceled)

24. The microvascular anastomotic coupler of claim 19, wherein the biodegradable sheath is configured to constrain the stent to a diameter of 1.5 mm.

25. The microvascular anastomotic coupler of claim 18, wherein the stent comprises an adhesive material selected from the group consisting of fibrin, cyanoacrylate, and photopolymerizable sealants.

26. The microvascular anastomotic coupler of claim 19, wherein the biodegradable sheath covers substantially the entire surface of the stent.

27. The microvascular anastomotic coupler of claim 19, wherein the biodegradable sheath comprises bioresorbable materials, polysaccharides or water-soluble polymers.

28. The microvascular anastomotic coupler of claim 18, wherein the member comprises water.

29. The microvascular anastomotic coupler of claim 27, wherein the biodegradable sheath comprises sucrose.

30. The microvascular anastomotic coupler of claim 19, wherein the biodegradable sheath is molded to the stent.

31. The microvascular anastomotic coupler of claim 28, wherein the member is molded to the stent.

32. The microvascular anastomotic coupler of claim 19, wherein the biodegradable sheath is a bioresorbable wrap.

33. The microvascular anastomotic coupler of claim 19, wherein the at least one member is a clasp.

34-45. (canceled)

46. A method of making an anastomotic coupler, the method comprising:
a) providing a mold assembly;
b) placing a material and a stent into the mold assembly; and
c) producing an anastomotic coupler comprising the stent and one or more sheaths covering the surface of the stent, whereby the one or more sheaths constrain the stent.

47-53. (canceled)
